SEQUENCE LISTING (6 sequences)

SEQ ID NO: 1 - DNA, 1194 bp, Rhesus
atgaactgcatatccgacttcttcacctatgaaaccaccaaatcggtggttgtgaagagctggaccatcgggatcatcaaccgagtagttcagcttctgatcatctcctactttgtggggtgggttttcttgcacgagaaggcataccaggtacgggacacagccattgagtcctcggtggtaaccaaggtgaagggcttcggactctacgccaacagagtcatggatgtgtctgattatgtgacgccacctcagggcacctcagtctttgtcatcatcaccaagatgattgttactgaaaaccagatgcaaggattctgcccagagagtgaggagaaataccgctgtgtatcagatagccagtgcgggcctgagcgcttcccaggcggggggatcctcactggccgctgcgtgaactacagctctgtgttccggacctgtgagatccagggctggtgccccacggaggtggacacggtggaaatgcccatcatgatggaagctgagaacttcactattttcatcaagaacagcatccgtttccccctcttcaactttgagaagggaaacctccttcccaacctgacagccagggacatgaagacctgccgcttccacccggacaaggaccctttctgccccatcttgcgggtaggggacgtggtcaagtttgcggggcaggattttgccaaactggcgcgcacgggtggcgttctgggcattaagattggctgggtgtgcgacttggacaaggcctgggaccagtgcatccccaagtactccttcacccggctggacagcgtttctgagaagagcagcgtgtccccaggctacaacttcaggtttgccaagtactacaaaatggagaatggcagtgagtaccgcaccctcctgaaggctttcggcatccgcttcgacgtgctggtgtacgggaatgctggcaagttcaacatcatccccaccatcatcagctctgtggcggcctttacttctgtgggagtggggactgttctctgtgacatcatcctgctcaacttcctcaagggggccgatcaatacaaggccaagaagtttgaggaggtgaatgagactacgctgaaaatcgccgctttgaccaacccagtgtaccccagcgaccagaccacggcggagaagcagtccactgactcgggggccttctccataggccactag

SEQ ID NO: 2 - protein, 397 aa, Rhesus
MNCISDFFTYETTKSVVVKSWTIGIINRVVQLLIISYFVGWVFLHEKAYQVRDTAIESSVVTKVKGFGLYANRVMDVSDYVTPPQGTSVFVIITKMIVTENQMQGFCPESEEKYRCVSDSQCGPERFPGGGILTGRCVNYSSVFRTCEIQGWCPTEVDTVEMPIMMEAENFTIFIKNSIRFPLFNFEKGNLLPNLTARDMKTCRFHPDKDPFCPILRVGDVVKFAGQDFAKLARTGGVLGIKIGWVCDLDKAWDQCIPKYSFTRLDSVSEKSSVSPGYNFRFAKYYKMENGSEYRTLLKAFGIRFDVLVYGNAGKFNIIPTIISSVAAFTSVGVGTVLCDIILLNFLKGADQYKAKKFEEVNETTLKIAALTNPVYPSDQTTAEKQSTDSGAFSIGH

SEQ ID NO: 3 - DNA, 25 bp, Rhesus
gaagcttaccatgaactgcatatcc

SEQ ID NO: 4 - DNA, 26 bp, Rhesus
gctcgagctagtggcctatggagaag

SEQ ID NO: 5 - DNA, 7 bp, Artificial Sequence
  PCR Primer
tgantca

SEQ ID NO: 6 - DNA, 11 bp, Artificial Sequence
  PCR Primer
ggggactttcc

(19) United States
(12) Patent Application Publication
(10) Pub. No.: US 2010/0221773 A1
(43) Pub. Date: Sep. 2, 2010

(54) Nucleic Acids Encoding a Functional Mammalin Purinoreceptor, P2X3, Methods of Production and Use Thereof

(76) Inventors: Stefanie A. Kane, Schwenksville, PA (US); John J. Mallee, Collegeville, PA (US); Christopher Salvatore, Philadelphia, PA (US); Jixin Wang, Wayne, PA (US)

Correspondence Address: MERCK, P O BOX 2000, RAHWAY, NJ, 07065-0907, US

(21) Appl. No.: 11/992,634

(22) PCT Filed: Sep. 26, 2006
(86) PCT No.: PCT/US06/37402

§ 371 Date: Mar. 26, 2008

Related U.S. Application Data
(60) Provisional application No. 60/722,231, filed on Sep. 30, 2005.

(51) Int. Cl. C12P 21/00; C07H 21/04; C12N 15/74; C12N 1/21; C12N 1/19; C12N 5/10

(52) U.S. Cl. 435/69.1; 536/23.5; 435/320.1; 435/252.3; 435/254.2; 435/325

(57) ABSTRACT

The subject invention relates to the rhesus monkey P₂X₃ receptor, nucleic acids encoding this receptor, methods of modulating the activity of a target purinergic receptor using the P₂X₃ receptor and to uses of these methods. In particular, such methods may be used, for example, to accelerate the rate of resensitization of a desensitized receptor.

### FIELD OF THE INVENTION

The invention relates generally to receptor proteins and to DNA and RNA molecules encoding therefor. In particular, the invention relates to a nucleic acid sequence that encodes a rhesus receptor P₂X₃. The invention also relates to methods of using the receptor encoded thereby to identify compounds that interact with it. This invention further relates to compounds which act as modulators, e.g., antagonists or agonists to compounds which have reactivity with the various P₂X receptor and methods utilized in determining said reactivity. The invention also involves therapeutic uses involving aspects of this receptor.

### BACKGROUND OF THE INVENTION

Purines, acting via an extracellular purinoreceptor, have been implicated as having a variety of physiological and pathological roles. (See, Burnstock (1993) Drug Dev. Res. 28:195-206.) Purinoreceptors (P₂) have been generally categorized as either metabotropic nucleotide receptors or ionotropic receptors for extracellular nucleotides. Metabotropic nucleotide receptors (usually designated P₂Y or P₂Y₍ₙ₎, where “n” is a subscript integer indicating subtype) are believed to differ from ionotropic receptors (usually designated P₂X or P₂X₍ₙ₎ in that they are based on a different fundamental means of transmembrane signal transduction: P₂Y receptors operate through a G protein-coupled system, while P₂X receptors are ligand-gated ion channels.

At least seven P₂X receptors, and the cDNA sequences encoding them, have been identified to date. P₂X₁ cDNA was cloned from the smooth muscle of the rat vas deferens (Valera et al. (1994) Nature 371:516-519) and P₂X₂ cDNA was cloned from PC12 cells (Brake et al. (1994) Nature 371:519-523). Five other P₂X receptors have been found in cDNA libraries by virtue of their sequence similarity to P₂X₁ and P₂X₂-P₂X₃: Lewis et al. (1995) Nature 377:432-435, Chen et al. (1995) Nature 377:428-431; P₂X₄: Buell et al. (1996) EMBO J. 15:55-62, Seguela et al. (1996) J. Neurosci. 16:448-455, Bo et al. (1995) FEBS Lett. 375:129-133, Soto et al. (1996) Proc. Natl. Acad. Sci. USA 93:3684-3688, Wang et al. (1996) Biochem. Biophys. Res. Commun. 220:196-202; P₂X₅: Collo et al. (1996) J. Neurosci. 16:2495-2507, Garcia-Guzman et al. (1996) FEBS Lett. 388:123-127; P₂X₆: Collo et al. (1996), supra, Soto et al. (1996) Biochem. Biophys. Res. Commun. 223:456-460; P₂X₇: Surprenant et al. (1996) Science 272:735-738). For a comparison of the amino acid sequences of rat P₂X receptor see Buell et al. (1996) Eur. J. Neurosci. 8:2221-2228.

Purinergic receptors, in particular, P₂X receptors, are known to function as homomultimeric cation-permeable ion channels and, in some cases, as heteromeric channels consisting of two different P₂X receptor subtypes (Lewis et al., Nature 377:432-435 (1995); Le et al., J. Neurosci. 18:7152-7159 (1998); Tones et al., Mol. Pharmacol. 54:989-993 (1998)). The P₂X₂ and P₂X₃ subunits form functional channels when expressed alone, and can also form a functional heteromultimeric channel that has properties similar to currents seen in native sensory channels when co-expressed. At least one pair of P₂X receptor subtypes, P₂X₂ and P₂X₃, functions as a heteromeric channel in rat nodose ganglion neurons where it exhibits distinct pharmacological and electrophysiological properties (Lewis et al., supra (1995)).

Native P₂X receptors are known to form rapidly activated, nonselective cationic channels upon activation by ATP. The channels formed by P₂X receptors generally have high Ca²⁺ permeability (P_{(Ca)}/P_{(Na)}). With respect to individual receptors, the P₂X₃ purinergic receptor is a ligand-gated cation channel that is selectively permeable to small cations. Known ligands for P₂X receptors include natural nucleotides, for example, ATP, UTP, UDP, or synthetic nucleotides, for example 2-methylthioATP. ATP, in addition to its function as an intracellular energy donor, is now recognized as an important neurotransmitter or cotransmitter, in both the central and peripheral nervous system (Ralevic, V., et al., Pharmacol. Rev., 50:413-492 (1998)). It is released from a variety of cell types, including nerve fibers, upon stimulation and produces diverse effects on many tissues by activation of specific membrane receptors including purinoreceptors (P2 receptor). See Burnstock, G., Pharmacol. Rev., 24:509-581 (1972); Burnstock, G., Cell Membrane Receptor for Drugs and Hormones: A Multidisciplinary Approach, edited by R. W. Straub and L. Bolid. New York: Raven, 1978, p. 107-118). With respect to the P₂X purinergic receptor, data suggest that ATP is capable of activating P₂X₃ homomeric receptors and P₂X₂/P₂X₃ heteromeric receptors where it functions as an excitatory neurotransmitter in the spinal cord dorsal horn and in primary afferents from sensory ganglia. In vitro, co-expression of P₂X₂ and P₂X₃ receptor subunits is necessary to produce ATP-gated currents with the properties seen in some sensory neurons. See, Lewis, et al. (1995) Nature 377:432-435.

ATP, and to a lesser extent, adenosine, can stimulate sensory nerve endings resulting in intense pain and a pronounced increase in sensory nerve discharge. According to available data, ATP released from damaged cells can evoke pain by activating P₂X₃ homomeric receptors, or P₂X₂/P₂X₃ heteromeric receptors expressed on nociceptive nerve endings of sensory nerves. This is consistent with reports of the induction of pain by intradermally applied ATP in the human blister-base model; the identification of P₂X₃ containing receptor on nociceptive neurons in the tooth pulp; and with reports that P₂X antagonists are analgesic in animal models. To date, research data suggests that the mechanism whereby ATP-induced activation of the P₂X purinergic receptors on dorsal root ganglion nerve terminals in the spinal cord and on neurons in the brain results in pain sensation is by the stimulation of the release of glutamate, a key neurotransmitter involved in nociceptive signaling.

It has also been recently demonstrated that P₂X₃ receptor gene disruption results in a diminished sensitivity to noxious chemical stimuli and reduced pain. The nociceptive effects of exogenously administered ATP and P₂X containing receptor agonists have also been demonstrated in laboratory animals. See Bland-Ward et al., Dr. J. Pharmacol. 122:366-371 (1997); Hamilton et al., Br. J. Phamacol. 126:326-332 (1999). The peripheral nociceptive actions of P₂X activation and stimulation of spinal P₂X containing receptor also contribute to nociception as indicated by the ability of intrathecally (i.t.) administered P₂ receptor agonists to increase sensitivity to acute and persistent noxious stimuli in rodents. See Driessen et al., Brain Res. 666:182-188 (1994); Tsuda et al., Br. J. Pharmacol. 127:449-4S6 (1999); Tsuda et al., Br. J. Pharmacol. 128:1497-1504 (1999). A selective P2 receptor-mediated increase in ectopic neuronal excitability that is localized to damaged sensory afferents has also been recently reported in rats following chronic constriction nerve injury. See Chen et al., NeuroReport 10:2779-2782 (1999). This role in pain transmission is consistent with the observation that the rat P₂X₃ receptor expression is found primarily in a subset of neurons of the sensory ganglia, which are involved in pain transmission. See Chen et al., Nature 377:428-430 (1995); Vulchanova et al., Neuropharmacol. 36:1229-1242 (1997).

Taken together, the functional and immunohistochemical localization of P₂X₃ containing receptors (P₂X₃ and/or P₂X₂/₃) on sensory nerves indicates that these P₂X receptors may have a primary role in mediating the nociceptive effects of exogenous ATP. Thus, compounds which block or inhibit activation of P₂X₃ receptors serve to block the pain stimulus. More, receptor antagonists to compounds which normally activate the P₂X₃ receptor and/or P₂X₂/P₂X₃ heteromeric channels, such as ATP, could successfully block the transmission of pain. Indeed, modulators of P₂X receptors, e.g., P₂X₃ receptor may find use as analgesics.

However, the utility of available purinergic ligands to evaluate the role of individual P₂ receptor subtypes in mammalian physiology has been complicated by the susceptibility of P₂ receptor agonists to undergo enzymatic degradation. As well, the study of the role of an individual P₂X receptor is hampered by the lack of receptor subtype-specific agonists and antagonists. For example, one agonist useful for studying ATP-gated channels is -methylene-ATP (αβ-meATP); however, the P₂X receptors display differential sensitivity to the agonist with P₂X₁ and P₂X₂ being αβ-meATP-sensitive and insensitive, respectively. Moreover, binding of αβ-meATP to P₂X receptors does not always result in channel opening. In addition, the predominant forms of P₂X receptor in the rat brain, P₂X₄ and P₂X₆ receptor, cannot be blocked by suramin or pyridoxal phosphate-6-azophenyl-2′,4′-disulphonic acid (“PPADS”). These two forms of the P₂X receptor are also not activated by a13-meATP and are thus intractable to study with currently available pharmacological tools.

Consequently, the state of the art begs an inquiry into methods which will provide the ability to regulate or control the P₂X receptors, for example, P₂X₃, because control of such receptors will provide the ability to minimize pain in patients in need of such treatment. In addition, for both research and therapeutic purposes there is a need in the art for specific agonists and antagonists for each P₂X receptor subtype and, in particular, agents that will be effective in vivo, as well as for methods for identifying purinoreceptor-specific agonist and antagonist compounds.

The present invention aims to overcome some of the aforementioned drawbacks by providing novel nucleic acid molecules and encoded proteins that may be used to identify potential purinoreceptor-specific modulators, that may play a critical role in treating disease states associated with pain, in particular peripheral pain, inflammatory pain, or tissue injury pain that can be treated using a P₂X₃ receptor subunit modulator. More specifically, the invention provides the tools necessary to effectively identify a potential purinoreceptor-specific antagonist that has the potential to act as a suitable analgesic.

### SUMMARY OF THE INVENTION

The present invention relates to a novel purinergic receptor, particularly a P₂X₃ type receptor, derived and isolated from the rhesus monkey.

In one embodiment, an isolated nucleic acid molecule, e.g., cDNA molecule or functionally active fragments thereof is provided, wherein the nucleic acid molecule encodes aforementioned rhesus P₂X₃ receptor, or functionally active subunits thereof.

In another embodiment, a recombinant vector comprising a nucleic acid molecule of the invention, or fragments thereof, is provided.

In another embodiment, the subject invention is directed to a rhesus P₂X₃ receptor polypeptide, either alone or in multimeric form.

In still other embodiments, the invention is directed to messenger RNA encoded by the nucleic acid molecule, recombinant host cells transformed or transfected with vectors comprising the nucleic acid molecule or fragments thereof, and methods of producing recombinant P₂X₃ polypeptides of the invention using such cells.

In yet another embodiment, the invention is directed to a method of expressing the above referenced rhesus P₂X₃ receptor, or a subunit thereof, in a cell to produce the resultant P₂X-containing receptor.

In a further embodiment, the invention is directed to a method of using such cells to identify potentially therapeutic compounds that modulate or otherwise interact with the above P₂X-containing receptor.

In another embodiment, therapeutic uses involving P₂X modulators, such as an ATP agonist or antagonist are contemplated.

In still another embodiment, the invention provides a method of identifying compounds that modulate a purinergic receptor or other therapeutic compounds using such cells. The method offers a variety of advantages, in that it (a) provides a means of distinguishing, during screening of compounds, between receptor agonists and antagonists; (b) exhibits greater sensitivity than conventional methodologies, especially with respect to P₂X receptor and known phosphoinositide hydrolysis assays; and/or (c) is suitable for testing all P₂ receptor agonists over a broad range of ligand concentrations.

In yet another embodiment, the invention provides a method for identifying compounds that modulate the activity of a purinoreceptor, particularly a P₂X purinoreceptor. The method comprises (a) providing a cell which is a purinoreceptor null cell in its native non-transformed state and which comprises and expresses a polynucleotide encoding a rhesus purinoreceptor polypeptide; (b) mixing a test compound with the cell; and (c) measuring either (i) the effect of the test compound on the activation of the rhesus purinoreceptor or the cell expressing the purinoreceptor receptor, or (ii) the binding of the test compound to the cell or the receptor.

An alternative embodiment provides a method for determining the amount of a receptor agonist or antagonist in a test sample. The method comprises (a) providing a cell that expresses a purinoreceptor polypeptide coding sequence, (b) mixing the recombinant or transformed cell with a test sample, and (c) measuring the effect of the test compound on the activation of the purinoreceptor or the cell expressing the purinoreceptor receptor.

The purinoreceptor so expressed and utilized for testing preferably comprises a P₂X receptor subunit, either alone or in combination with another subunit.

These and other embodiments of the present invention will readily occur to those of ordinary skill in the art in view of the disclosure herein. In particular, while The description and examples which follow are drawn to the cloning, expression and testing of rhesus purinoreceptor, it is anticipated that the method of the present invention may be carried out using analogous mammalian receptor from non-rhesus sources, whose sequences and/or functional properties are similar enough to those of the corresponding rhesus receptor as to be readily substituted therefor without undue experimentation. Such non-rhesus, and especially rat and human, P₂ receptor clones may in such instances be regarded as equivalents to their rhesus counterparts. The structure and chromosomal mapping of mouse genomic P₂X₃ receptor subunit has also been described. See, Souslova, et al. (1997) Gene 195:101-111.

### BRIEF DESCRIPTION OF THE DRAWINGS

FIG. 1 depicts a dose-dependent blockade of αβ-meATP activated Ca²⁺ influx by PPADS on the rhesus P₂X₃ receptor of the invention. Cells were incubated with PPADS for 10 min prior to the addition of agonist. Overlay of raw FLIPR traces obtained from a representative experimental run shows dose-dependent inhibition of fluorescence change by PPADS.

FIG. 2 depicts A) a representative rhesus P₂X₃ inward current evoked by 30 μM αβ-meATP; αβ-meATP dose-response curve for rhesus P₂X₃ receptor

FIG. 3 depicts A) a representative experiment in which the rhesus P₂X₃ inward current elicited by 100 mM CTP (control) is diminished in the presence of 3 μM PPADS; B) PPADS dose-response curve for PPADS on the rhesus P₂X₃ receptor

FIG. 4 depicts the nucleotide sequence (SEQ ID NO.:1) of the novel purinergic receptor of the invention.

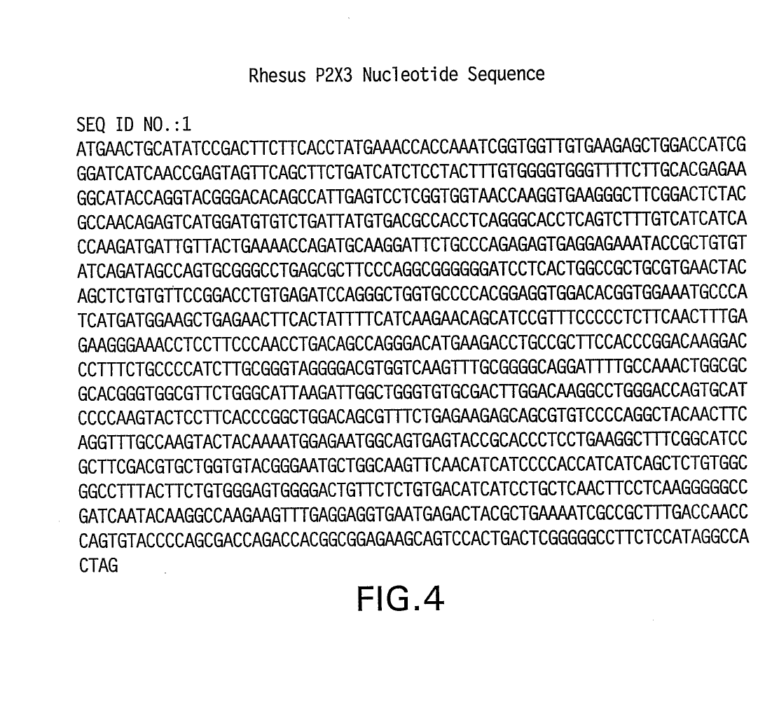

FIG. 5 depicts the deduced amino acid sequence (SEQ ID NO.: 2) of the P₂X₃ receptor of the invention.

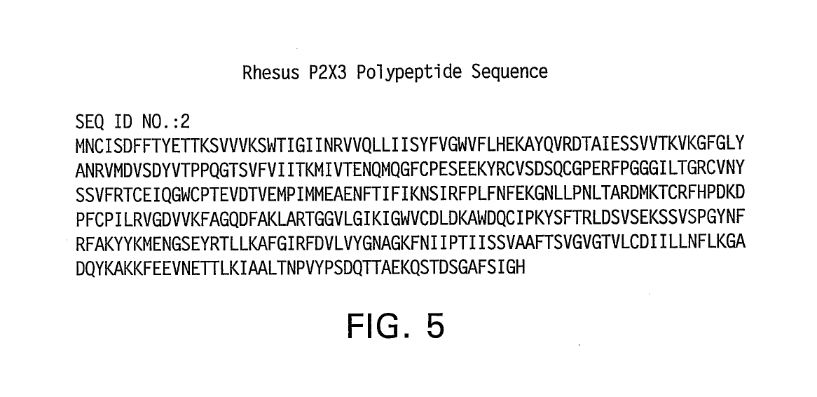

### DETAILED DESCRIPTION OF THE INVENTION

As used in this specification and the appended claims, the singular forms “a”, “loan” and “the” include plural references unless the content clearly dictates otherwise.

Practice of the present invention will employ, unless otherwise indicated, conventional techniques of molecular biology, microbiology, recombinant DNA technology, electrophysiology, and pharmacology, that are within the skill of the art. Such techniques are explained fully in the literature. See, for example, Sambrook, Fritsch & Maniatis, Molecular Cloning: A Laboratory Manual, Second Edition (1989); DNA Cloning, Vols. I and II (D. N. Glover Ed. 1985); Perbal, B., A Practical Guide to Molecular Cloning (1984); the series, Methods In Enzymology (S. Colowick and N. Kaplan eds., Academic Press, Inc.); Transcription and Translation (Hames et al. eds. 1984); Gene Transfer Vectors For Mammalian Cells (J. H. Miller et al. eds. (1987) Cold Spring Harbor Laboratory, Cold Spring Harbor, N.Y.); Scopes, Protein Purification: Principles and Practice (2nd ed., Springer-Verlag); and PCR: A Practical Approach (McPherson et al. eds. (1991) IRL Press).

All patents, patent applications and publications cited herein, whether supra or infra, are hereby incorporated by reference in their entirety and are deemed representative of the prevailing state of the art.

As used in this specification and the appended claims, the singular forms “a,” “an” and “the” include plural references unless the content clearly dictates otherwise. Thus, for example, reference to “a primer” includes two or more such primers, reference to “an amino acid” includes more than one such amino acid, and the like.

### A. DEFINITIONS

In describing the present invention, the following terms will be employed, and are intended to be defined as indicated below.

The terms “P₂ receptor” intended to include a purinergic receptor for the ligand ATP and/or other purine or pyrimidine nucleotides, natural or synthetic. P₂ receptor are broadly subclassified as “P₂X” or “P₂Y” receptor. These types differ in their pharmacology, structure, and signal transduction mechanisms. The P₂X receptor are generally ligand-gated ion channels, while the P₂Y receptor operate generally through a G protein-coupled system. Moreover, and unless otherwise specified, the term “P₂ receptor” is further intended to mean both homomeric (consisting of one or more identical subunits) and heteromeric (comprising two or more different P₂X subunits) receptor. Without intending to be limited by theory, such homomers and heteromers are believed to exist in vivo but may also be expressed by the cloning and transfection methods described below.

The term “subunit” when used in reference to purinoreceptor intends a polypeptide which, either alone or in combination with one or more other polypeptides, forms a functional purinoreceptor. Where a purinoreceptor comprises more than one polypeptide subunit, the subunits may be either identical (forming a homomeric multimer) or different (forming a heteromeric multimer).

“P₂X₃ receptor subunit” refers to the amino acid sequences of substantially purified P₂X₃ receptor subunit obtained from any species particularly mammalian species, more particularly the rhesus P₂X₃, from any source, whether natural, synthetic, semi-synthetic, or recombinant.

As used herein, an “agonist” refers to a ligand that activates an intracellular response when it binds to a receptor at concentrations equal or lower to the concentrations of a known ligand which induce an intracellular response. Thus, an agonist according to the invention may increase the intracellular response mediated by a receptor by at least 2-fold, preferably 5-fold, more preferably 10-fold and most preferably 100-fold or more (i.e., 150-fold, 200-fold, 250-fold, 500-fold, 1000-fold, 10,000-fold etc.), as compared to the intracellular response in the absence of agonist. For example, in the case of P₂X₃, the natural ligand may be ATP such that an agonist may increase the concentrations of cystolic calcium two (2) fold compared to that achieved when ATP activates endogenous P₂X₃. As well, P₂X₃ agonist may also refer to a molecule, which, when bound to P₂X receptor complex, or is within proximity of P₂X receptor complex, modulates the activity of P₂X receptor complex by increasing or prolonging the duration of the effect of P₂X receptor complex. Agonists can include nucleotides, proteins, nucleic acids, carbohydrates, organic compounds, inorganic compounds, or any other molecules which modulate the effect of P₂X receptor complex, in particular the P₂X₃ receptor subunit of the invention, e.g., rhesus P₂X₃. Preferably, a “P₂X₃ receptor agonist” is a compound that binds to and activates a P₂X₃ receptor. By “activates” is intended the elicitation of one or more pharmacological, physiological, or electrophysiological responses. Such a response includes, but is not limited to, an increase in receptor-specific cellular depolarization or increase in intracellular calcium levels due to calcium ion influx for a P₂Xₙ receptor, or an increase in intracellular concentration of free Ca²⁺ ([Ca.sup.2+]ᵢ) and/or inositolphospholipid hydrolysis and the formation of inositol phosphate for a P₂Yₙ receptor. An “inhibitor” compound according to the invention is a molecule directed against the receptor, e.g., P₂X₃ or against the natural ligand for the receptor that decreases the binding of the ligand to the receptor by at least 10%, preferably 15-25%, more preferably 25-50% and most preferably, 50-100%, in the presence of ATP, as compared to the binding in the presence of ATP and in the absence of inhibitor. An “inhibitor” compound of the invention can decrease the intracellular response induced by an agonist, for example ATP, by at least 10%, preferably 15-25%, more preferably 25-50% and most preferably, 50-100%. An “inhibitor” also refers to a nucleotide sequence encoding an inhibitor compound of the invention.

As used herein, a “modulator” refers to any compound that increases or decreases the cell surface expression of a receptor of the invention, increases or decreases the binding of a ligand to a receptor of the invention, or any compound that increases or decreases the intracellular response initiated by an active form of the receptor of the invention, either in the presence or absence of an agonist, and in the presence of a ligand for the receptor, for example ATP. A modulator includes an agonist, antagonist, inhibitor or inverse agonist, as defined herein. A modulator can be a protein, a nucleic acid, an antibody or fragment thereof, a peptide, etc. Candidate modulators can be natural or synthetic compounds, including, for example, small molecules, compounds contained in extracts of animal, plant, bacterial or fungal cells, as well as conditioned medium from such cells.

“Nucleic acid” and “nucleic acid sequence” or “nucleic acid molecule” or equivalent terms thereof refer to a nucleotide, oligonucleotide, polynucleotide, or any fragment thereof. These phrases also refer to DNA or RNA of genomic or synthetic origin which may be single stranded or doublestranded and may represent the sense or the antisense strand, a peptide nucleic acid (PNA), or any DNA-like or RNA-like material. In this context, “fragments” refer to those nucleic acids which, when translated, would produce polypeptides retaining some functional characteristic, e.g., antigenicity, or structural domain, e.g., ion channel domain, characteristic of the full-length polypeptide. Likewise, the term “polynucleotide” as used herein means a polymeric form of nucleotides of any length, either ribonucleotides or deoxyribonucleotides. This term refers only to the primary structure of the molecule. Thus, the term includes double- and single-stranded DNA, as well as double- and single-stranded RNA. It also includes modifications, such as by methylation and/or by capping, and unmodified forms of the polynucleotide.

An “oligonucleotide” refers to a nucleic acid molecule of at least about 6 to 50 nucleotides, preferably about 15 to 30 nucleotides, and more preferably 20 to 25 nucleotides, which can be used in PCR amplification or in a hybridization assay. “Oligonucleotide” is substantially equivalent to the terms “amplimer”, “primer”, “oligomer”, and “probe” as these terms are commonly defined in the art.

The term “variant” is used to refer to an oligonucleotide sequence which differs from the related wild-type sequence in the insertion, deletion or substitution of one or more nucleotides. When not caused by a structurally conservative mutation (see below), such a variant oligonucleotide is expressed as a “protein variant” which, as used herein, indicates a polypeptide sequence that differs from the wild-type polypeptide in the insertion, deletion or substitution of one or more amino acids. The protein variant differs in primary structure (amino acid sequence), but may or may not differ significantly in secondary or tertiary structure or in function relative to the wild-type.

The term “mutant” generally refers to an organism or a cell displaying a new genetic character or phenotype as the result of change in its gene or chromosome. In some instances, however, “mutant” may be used in reference to a variant protein or oligonucleotide and “mutation” may refer to the change underlying the variant.

“Polypeptide” and “protein” are used interchangeably herein and indicate a molecular chain of amino acids linked through peptide bonds. The terms do not refer to a specific length of the product. Thus, peptides, oligopeptides, and proteins are included within the definition of polypeptide. The terms include post-translational modifications of the polypeptide, for example, glycosylations, acetylations, phosphorylations and the like. In addition, protein fragments, analogs, mutated or variant proteins, fusion proteins and the like are included within the meaning of polypeptide, provided that such fragments, etc. retain the binding or other characteristics necessary for their intended use. For example, a “P₂Xₙ” or “purinergic receptor polypeptide” generally refers to the novel P₂X₃ receptor polypeptide comprising the amino acid sequence of SEQ ID NO.:2 of functionally active fragments or mutants thereof.

A “functionally conservative mutation” as used herein intends a change in a polynucleotide encoding a derivative polypeptide in which the activity is not substantially altered compared to that of the polypeptide from which the derivative is made. Such derivatives may have, for example, amino acid insertions, deletions, or substitutions in the relevant molecule that do not substantially affect its properties. For example, the derivative can include conservative amino acid substitutions, such as substitutions which preserve the general charge, hydrophobicity/hydrophilicity, side chain moiety, and/or steric bulk of the amino acid substituted, for example, Gly/Ala, Val/Ile/Leu, Asp/Glu, Lys/Arg, Asn/Gln, Thr/Ser, and Phe/Trp/Tyr.

By the term “structurally conservative mutant” is intended a polynucleotide containing changes in the nucleic acid sequence but encoding a polypeptide having the same amino acid sequence as the polypeptide encoded by the polynucleotide from which the degenerate variant is derived. This can occur because a specific amino acid may be encoded by more than one “codon,” or sequence of three nucleotides, i.e., because of the degeneracy of the genetic code.

“Recombinant host cells”, “host cells,” “cells,” “cell lines,” “cell cultures,” and other such terms denoting cell lines cultured as unicellular entities refer to cells which can be, or have been, used as recipients for recombinant vectors or other transfer DNA, immaterial of the method by which the DNA is introduced into the cell or the subsequent disposition of the cell. The terms include the progeny of the original cell which has been transfected. Cells in primary culture as well as cells such as oocytes also can be used as recipients.

A “vector” is a replicon in which another polynucleotide segment is attached, such as to bring about the replication and/or expression of the attached segment. The term includes expression vectors, cloning vectors, and the like.

A “coding sequence” is a polynucleotide sequence that is transcribed into mRNA and/or translated into a polypeptide. The boundaries of the coding sequence are determined by a translation start codon at the 5′-terminus and a translation stop codon at the 3′-terminus. A coding sequence can include, but is not limited to, mRNA, cDNA, and recombinant polynucleotide sequences. Variants or analogs may be prepared by the deletion of a portion of the coding sequence, by insertion of a sequence, and/or by substitution of one or more nucleotides within the sequence. Techniques for modifying nucleotide sequences, such as site-directed mutagenesis, are well known to those skilled in the art. See, for example, Sambrook et al., supra; DNA Cloning, Vols. I and II, supra; Nucleic Acid Hybridization, supra.

“Operably linked” refers to a situation wherein the components described are in a relationship permitting them to function in their intended manner. Thus, for example, a control sequence “operably linked” to a coding sequence is ligated in such a manner that expression of the coding sequence is achieved under conditions compatible with the control sequences. A coding sequence may be operably linked to control sequences that direct the transcription of the polynucleotide whereby said polynucleotide is expressed in a host cell. The term “control elements” refers collectively to promoters, ribosome binding sites, polyadenylation signals, transcription termination sequences, upstream regulatory domains, enhancers, and the like, which collectively provide for the transcription and translation of a coding sequence in a host cell. Not all of these control sequences need always be present in a recombinant vector so long as the desired gene is capable of being transcribed and translated.

A “reporter gene” is a gene that, upon expression, confers a phenotype on a cell expressing the reporter gene, such that the cell can be identified under appropriate conditions. For example, the reporter gene may produce a polypeptide product that can be easily detected or measured in a routine assay. Suitable reporter genes known in the art which confer this characteristic include those that encode chloramphenicol acetyl transferase (CAT activity), β-galactosidase, luciferase, alkaline phosphatase, human growth hormone, fluorescent proteins, such as green fluorescent protein (GFP), and others. Indeed, any gene that encodes a protein or enzyme that can readily be measured, for example, by an immunoassay such as an enzyme-linked immunosorbent assay (ELISA) or by the enzymatic conversion of a substrate into a detectable product, and that is substantially not expressed in the host cells (specific expression with no background) can be used as a reporter gene to test for promoter activity. Other reporter genes for use herein include genes that allow selection of cells based on their ability to thrive in the presence or absence of a chemical or other agent that inhibits an essential cell function. Suitable markers, therefore, include genes coding for proteins which confer drug resistance or sensitivity thereto, or change the antigenic characteristics of those cells expressing the reporter gene when the cells are grown in an appropriate selective medium. For example, reporter genes include: cytotoxic and drug resistance markers, whereby cells are selected by their ability to grow on media containing one or more of the cytotoxins or drugs; auxotrophic markers by which cells are selected by their ability to grow on defined media with or without particular nutrients or supplements; and metabolic markers by which cells are selected for, e.g., their ability to grow on defined media containing the appropriate sugar as the sole carbon source. These and other reporter genes are well known in the art.

A “change in the level of reporter gene product” is shown by comparing expression levels of the reporter gene product in a cell exposed to a candidate compound relative to the levels of reporter gene product expressed in a cell that is not exposed to the test compound and/or to a cell that is exposed to a control compound. The change in level can be determined quantitatively for example, by measurement using a spectrophotometer, spectrofluorometer, lurninorneter, and the like, and will generally represent a statistically significant increase or decrease in the level from background. However, such a change may also be noted without quantitative measurement simply by, e.g., visualization, such as when the reporter gene is one that confers the ability of cells to form colored colonies on chromogenic substrates, or the ability of cells to thrive and/or die in the presence of test compounds.

The term “phenotype” refers to the physical, biochemical, and physiological makeup of an animal as determined both genetically and environmentally. As used herein, the phrase “resulting phenotype” refers to a wild-type animal or an animal having a disease state associated with a genitourinary disorder treated with a potential therapeutic compound which results in a phenotype similar to that of a KO animal.

The term “expression” as used herein intends both transcriptional and translational processes, i.e., the production of messenger RNA and/or the production of protein therefrom. “Subject” or “animal” means mammals and non-mammals. Mammals means any member of the Mammalia class including, but not limited to, humans, non-human primates such as chimpanzees and other apes and monkey species; farm animals such as cattle, horses, sheep, goats, and swine; domestic animals such as rabbits, dogs, and cats; laboratory animals including rodents, such as rats, mice, and guinea pigs; and the like. Examples of non-mammals include, but are not limited to, birds, reptiles, and the like. The term “subject” does not denote a particular age or sex.

The term “transformed” refers to any known method for the insertion of foreign DNA or RNA sequences into a host prokaryotic cell. The term “transfected” refers to any known method for the insertion of foreign DNA or RNA sequences into a host eukaryotic cell. Such transformed or transfected cells include stably transformed or transfected cells in which the inserted DNA is rendered capable of replication in the host cell. They also include transiently expressing cells which express the inserted DNA or RNA for limited periods of time. The transformation or transfection procedure depends on the host cell being transformed. It can include packaging the polynucleotide in a virus as well as direct uptake of the polynucleotide, such as, for example, lipofection, electroporation, or microinjection. Transformation and transfection can result in incorporation of the inserted DNA into the genome of the host cell or the maintenance of the inserted DNA within the host cell in plasmid form. Methods of transformation are well known in the art and include, but are not limited to, viral infection, electroporation, lipofection, and calcium phosphate mediated direct uptake.

The term “transfection” refers to the insertion of an exogenous polynucleotide into a host cell, irrespective of the method used for the insertion, or the molecular form of the polynucleotide that is inserted. The insertion of a polynucleotide per se and the insertion of a plasmid or vector comprised of the exogenous polynucleotide are included. The exogenous polynucleotide may be directly transcribed and translated by the cell, maintained as a nonintegrated vector, for example, a plasmid, or alternatively, may be stably integrated into the host genome. “Transfection” generally is used in reference to a eukaryotic cell while the term “transformation” is used to refer to the insertion of a polynucleotide into a prokaryotic cell. “Transformation” of a eukaryotic cell also may refer to the formation of a cancerous or tumorigenic state.

The term “isolated,” when referring to a polynucleotide or a polypeptide, intends that the indicated molecule is present in the substantial absence of other similar biological macromolecules. The term “isolated” as used herein means that at least 75 wt. %, more preferably at least 85 wt. %, more preferably still at least 95 wt. %, and most preferably at least 98 wt. % of a composition is the isolated polynucleotide or polypeptide. An “isolated polynucleotide” that encodes a particular polypeptide refers to a polynucleotide that is substantially free of other nucleic acid molecules that do not encode the subject polypeptide; however, the molecule may include functionally and/or structurally conservative mutations as defined herein.

A “test sample” as used herein intends a component of an individual's body which is a source of one of the P₂X receptor, including P₂X₃ and P₂X₆. These test samples include biological samples which can be evaluated by the methods of the present invention described herein and include body fluids such as whole blood, tissues and cell preparations.

The following single-letter amino acid abbreviations are used throughout the text:

Alanine A Arginine R.

Asparagine N Aspartic acid D

Cysteine C Glutamine Q

Glutamic acid E Glycine G

Histidine H Isoleucine 1

Leucine L Lysine K

Methionine M Phenylalanine F
Proline P Serine S
Threonine T Tryptophan W
Tyrosine Y Valine V

A “null” host cell is a host cell that does not express a purinoreceptor of interest within the limits of the methods used to measure the expression of such receptor, either pharmacologically, electrophysiologically, biochemically, or the like. Thus, a P₂X-P₂Y null cell, that is, a “P₂X⁻P₂Y⁻ host cell,” is a cell in which the presence of neither the P₂X receptor nor the P₂Y receptor can be measured. This characteristic of the null host cell may be due to mutation of a gene that otherwise naturally encodes a P₂X or P2Y receptor such that the mutant encodes a purinoreceptor polypeptide that is not detectable by methods used to detect the native P₂X or P₂Y receptor. On the other hand, a null host cell may not express a purinoreceptor polypeptide to any measurable extent. The definition of “null” host cell is not intended to be limited to any particular mechanism underlying the absence of measurable levels of a purinoreceptor.

The phrase “correlates with expression of a polynucleotide” refers to the detection of the presence of nucleic acids, the same or related to a nucleic acid sequence encoding P₂X₃ receptor subunit, e.g., by northern analysis or RT-PCR, is indicative of the presence of nucleic acids encoding P₂X₃ receptor subunit in a sample, and thereby is indicative of the expression of the transcript from the polynucleotide encoding P₂X₃ receptor subunit.

The phrase “detectably labeled” as used herein means joining, either covalently or non-covalently to the polynucleotides, polypeptides, or antibodies of the present invention, a substance which provides for a detectable signal. A wide variety of labels and conjugation techniques are well known in the art. Suitable labels include radionuclides, e.g., ³²P, ³⁵S, 3H, enzymes, substrates, cofactors, inhibitors, fluorescent moieties, chemiluminescent moieties, magnetic particles, and the like.

The phrase “disease state” means any disease, condition, symptom, disorder, or indication mediated by a purinoreceptor, more specifically a purinergic receptor such as a P₂X₃ receptor subtype.

The phrases “effective amount” or “therapeutically effective amount” mean a concentration of P₂X receptor complex modulator sufficient to inhibit or enhance the effect of the P₂X receptor complex.

The term “modulate” refers to a change in the activity of P₂X receptor complex which contains at least one P₂X₃ receptor subunit. For example, modulation may cause an increase or a decrease in protein activity, binding characteristics, ion channel opening and conductance, receptor or second messenger signaling, or any other biological, functional, or immunological properties of P₂X receptor complex. The term “modulator” is a composition having the ability to effect the above changes. The ability to modulate the activity of P₂X₃ receptor can be exploited in assays to screen for organic, inorganic, or biological compounds which affect the above properties of P₂X receptor complex, in particular the P₂X₃ receptor subunit.

“Nociceptor” refers to a receptor for pain caused by injury to body tissues; the injury may be from physical stimuli such as mechanical, thermal, or electrical stimuli, or from chemical stimuli such as the presence of a toxin or an excess of a non-toxic substance, e.g., ATP or formalin. The term “nociception” refers to the sensing of pain through these receptors.

“Nocifensor” refers to a system of nerves in the skin and mucous membranes which are concerned with local defense against injury. “Nocifensive behavior” refers to pain related behavior in response to mechanical, thermal, electrical, or chemical stimuli, e.g., hind paw lifting in rats.

“Pain” means the more or less localized sensation of discomfort, distress, or agony, resulting from the stimulation of specialized nerve endings. There are many types of pain, including, but not limited to, lightning pains, phantom pains, shooting pains, acute pain, inflammatory pain, neuropathic pain, complex regional pain, neuralgia, neuropathy, tissue injury pain, and the like (Dorland's Illustrated Medical Dictionary, 28th Edition, W.B. Saunders Company, Philadelphia, Pa.). The goal of treatment of pain is to reduce the degree or severity of pain perceived by a treatment subject.

“Treating” or “treatment of” a disease state includes: 1) preventing the disease state, i.e. causing the clinical symptoms of the disease state not to develop in a subject that may be exposed to or predisposed to the disease state, but does not yet experience or display symptoms of the disease state; 2) inhibiting the disease state, i.e., arresting the development of the disease state or its clinical symptoms; 3) or relieving the disease state, i.e., causing temporary or permanent regression of the disease state or its clinical symptoms.

### B. GENERAL METHODS

The subject invention relates to a novel purinergic P₂X receptor subtype, specifically the P₂X₃ receptor polypeptide derived from a rhesus monkey. In particular, the invention provides the nucleic acid sequence encoding this receptor, the amino acid sequence of the receptor, methods of producing this receptor, together with various methods of using the receptor including methods of screening for modulators of this receptor.

The ability to externally regulate the receptor may allow one, for example, to control sensations such as pain, following a traumatic accident, during the course of a terminal illness, during surgery, after surgery or during any situation during which a patient's pain must be managed by a medical provider.

In particular, the present invention provides a method for screening a plurality of compounds for specific binding to a purinoreceptor to identify a compound that modulates the activity of the receptor. The method comprises (a) providing a cell that expresses the human (or other mammalian) purinoreceptor polypeptide coding sequence, (b) mixing a test compound with the cell, and (c) measuring the effect of the test compound on the activation of the purinoreceptor or the cell expressing the purinoreceptor receptor.

In addition, the invention provides a method for determining the amount of a receptor agonist or antagonist in a test sample. The method comprises (a) providing a cell that expresses the rhesus (or other mammalian) purinoreceptor polypeptide coding sequence, (b) mixing the cell with a test sample, and (c) measuring the effect of the test compound on the activation of the purinoreceptor or the cell expressing the purinoreceptor receptor.

The invention also encompasses a host cell that encodes the purinoreceptor of interest. The host cell is genetically engineered with a vector, also encompassed by the present invention, which may be a cloning vector or an expression vector. The vector comprises a polynucleotide sequence encoding a purinoreceptor operably linked to control sequences that control its expression. Preferably, the host cell is stably transfected to express the purinoreceptor. More preferably, the host cell is a purinoreceptor null cell which, if not already lacking endogenous purinoreceptor expression, has been so engineered.

Rhesus P₂X₃ receptor, polynucleotide encoding variant receptor or polypeptide subunit thereof, and methods of making these receptors are provided herein. The invention includes not only the above P₂X receptor but also methods for screening compounds using the receptor and cells expressing the receptor. Further, polynucleotides and antibodies which can be used in methods for detection of the receptor, as well as the reagents useful in these methods, are provided. Compounds and polynucleotides useful in regulating the receptor and its expression also are provided as disclosed herein below.

In one preferred embodiment, the polynucleotide encodes the aforementioned rhesus P₂X receptor polypeptides or protein variants thereof containing conservative amino acid substitutions.

DNA encoding the target purinergic P₂X receptor of the invention, and variants thereof, can be derived from genomic or cDNA, prepared by synthesis, or by a combination of techniques. The DNA can then be used to express the rhesus P₂X receptor or as a template for the preparation of RNA using methods well known in the art (see, Sambrook et al., supra), or as a molecular probe capable of selectively hybridizing to, and therefore detecting the presence of, other P₂X-encoding nucleotide sequences.

cDNA encoding a P₂Xₙ receptor, e.g., the rhesus monkey P₂X₃ receptor may be obtained from an appropriate DNA library. cDNA libraries may be probed using the procedure described by Grunstein et al. (1975) Proc. Natl. Acad. Sci. USA 73:3961. The cDNA thus obtained can then be modified and amplified using the polymerase chain reaction (“PCR”) and primer sequences to obtain the DNA encoding the desired P₂Xₙ or P₂Yₙ receptor. Alternatively, the wild-type DNA may be obtained from an appropriate DNA library. DNA libraries may be probed using the procedure described by Grunstein et al. (1975) Proc. Natl. Acad. Sci. USA 73:3961. The wild-type cDNA thus obtained can then modified and amplified using PCR and mutated primer sequences to obtain the DNA encoding the receptor of interest.

More particularly, PCR employs short oligonucleotide primers (generally 10-20 nucleotides in length) that match opposite ends of a desired sequence within the DNA molecule. The sequence between the primers need not be known. The initial template can be either RNA or DNA. If RNA is used, it is first reverse transcribed to cDNA. The cDNA is then denatured, using well known techniques such as heat, and appropriate oligonucleotide primers are added in molar excess.

Primer extension is effected using DNA polymerase in the presence of deoxynucleotide triphosphates or nucleotide analogs. The resulting product includes the respective primers at their 5′-termini, covalently linked to the newly synthesized complements of the original strands. The replicated molecule is again denatured, hybridized with primers, and so on, until the product is sufficiently amplified. Such PCR methods are described in for example, U.S. Pat. Nos. 4,965,188; 4,800,159; 4,683,202; 4,683,195; incorporated herein by reference in their entireties. The product of the PCR is cloned and the clones containing the P₂X receptor DNA, derived by segregation of the primer extended strand, selected. Selection can be accomplished using a primer as a hybridization probe.

Alternatively, DNA encoding a rhesus (or other mammalian) P₂X₃ receptor can be derived from genomic or cDNA, prepared by synthesis, or by a combination of techniques. The DNA can then be used to express the receptor or as a template for the preparation of RNA using methods well known in the art (see, Sambrook et al., supra). An example of a method for obtaining the desired DNA involves isolating cDNA encoding the wild-type rhesus P₂Y₂ receptor as described by Parr et al. (1994), supra.

Alternatively still, the P₂Xₙ receptor, P₂X₃ receptor encoding DNA could be generated using an RT-PCR (reverse transcriptase—polymerase chain reaction) approach starting with RNA. The RNA may be obtained from cells or tissue in which the desired P₂Xₙ receptor is expressed, e.g., brain, spinal cord, uterus or lung, using conventional methods. For example, single-stranded cDNA is synthesized from RNA as the template using standard reverse transcriptase procedures and the cDNA is amplified using PCR. This is but one example of the generation of a P₂Xₙ receptor from a mammalian tissue RNA template.

Reverse transcription of rhesus RNAs can also be accomplished utilizing reagents from the Superscript Preamplification System (Invitrogen, Carlsbad, Calif.) and the following method: Poly A+ RNA (1 microgram) derived from pituitary gland tissue (BD Biosciences, Mountain View, Calif.) and 1.mu.1 (50 nanograms) random hexamer primers are combined in a final volume of 12.mu.1 dH.sub.2 O. This mixture is heated to 70.degree. C. for 10 minutes and chilled on ice for 1 minute. The following components are added: 2.mu.1 10×PCR buffer (200 mM Tris-HCl pH 8.4, 500 mM KCl), 2.mu.1 25 mM MgCl.sub.2, 1.mu.1 10 mM dNTP mix, and 2.mu.1 0.1M dithiothreitol. The reaction is equilibrated for 5 minutes at 25.degree. C. after which 1.mu.1 (200 units) Superscript II reverse transcriptase is added and incubation continued at 25.degree. C. for 10 minutes, followed by 50 minutes at 42.degree. C. Alternatively, 10 picomoles Oligo dT primer can be substituted for the random hexamer primers in the above reaction mixture. In this case, equilibration is carried out at 42.degree. C. for 2 minutes after which the reverse transcriptase is added and incubation continued at 42.degree. C. for 50 minutes. The reverse transcription reaction is terminated by incubation at 70.degree. C. for 15 minutes and chilled on ice. Rnase H (1.mu.1; 2 units) is added and the mixture incubated for 20 minutes at 37.degree. C., then stored on ice.

Synthetic oligonucleotides may be prepared using an automated oligonucleotide synthesizer such as that described by Warner (1984) DNA 3:401. If desired, the synthetic strands may be labeled with .sup.32 P by treatment with polynucleotide kinase in the presence of .sup.32 P-ATP, using standard conditions for the reaction. DNA sequences, including those isolated from genomic or cDNA libraries, may be modified by known methods which include site-directed mutagenesis as described by Zoller (1982) Nucleic Acids Res. 10:6487. Briefly, the DNA to be modified is packaged into phage as a single stranded sequence, and converted to a double stranded DNA with DNA polymerase using, as a primer, a synthetic oligonucleotide complementary to the portion of the DNA to be modified, and having the desired modification included in its own sequence. Culture of the transformed bacteria, which contain replications of each strand of the phage, are plated in agar to obtain plaques. Theoretically, 50% of the new plaques contain phage having the mutated sequence, and the remaining 50% have the original sequence. Replicates of the plaques are hybridized to labeled synthetic probe at temperatures and conditions suitable for hybridization with the correct strand, but not with the unmodified sequence. The sequences which have been identified by hybridization are recovered and cloned. Alternatively, it may be necessary to identify clones by sequence analysis if there is difficulty in distinguishing the variant from wild-type by hybridization. In any case, the DNA would be sequence-confirmed.

Once produced, DNA encoding the target P₂Xₙ purinoreceptor, e.g., rhesus P₂X₃ may then be incorporated into a cloning vector or an expression vector for replication in a suitable host cell. Vector construction employs methods known in the art. Generally, site-specific DNA cleavage is performed by treating with suitable restriction enzymes under conditions that generally are specified by the manufacturer of these commercially available enzymes. After incubation with the restriction enzyme, protein is removed by extraction and the DNA recovered by precipitation. The cleaved fragments may be separated using, for example, polyacrylamide or agarose gel electrophoresis methods, according to methods known by those of skill in the art.

Sticky end cleavage fragments may be blunt ended using E. coli DNA polymerase 1 (Klenow) in the presence of the appropriate deoxynucleotide triphosphates (dNTPs) present in the mixture. Treatment with S1 nuclease also may be used, resulting in the hydrolysis of any single stranded DNA portions.

Ligations are performed using standard buffer and temperature conditions using T4 DNA ligase and ATP. Alternatively, restriction enzyme digestion of unwanted fragments can be used to prevent ligation.

An alternative embodiment provides a host cell that encodes the purinoreceptor of interest. The host cell is genetically engineered with a vector, which may be a cloning vector or an expression vector, comprising a polynucleotide sequence encoding a purinoreceptor operably linked to control sequences that control its expression. Preferably, the host cell is stably transfected to express the purinoreceptor. In one embodiment the host cell may be a purinoreceptor null cell which, if not already lacking endogenous purinoreceptor expression, has been so engineered.

The vector may be in the form of a plasmid, a viral particle, a phage, etc. The engineered host cells can be cultured in conventional nutrient media modified as appropriate for activating promoters, selecting transformants/transfectants or amplifying the subunit-encoding polynucleotide. The culture conditions, such as temperature, pH and the like, generally are similar to those previously used with the host cell selected for expression, and will be apparent to those of skill in the art.

Standard vector constructions generally include specific antibiotic resistance elements. Ligation mixtures are transformed into a suitable host, and successful transformants selected by antibiotic resistance or other markers. Plasmids from the transformants can then be prepared according to methods known to those in the art usually following a chloramphenicol amplification as reported by Clewell et al. (1972) J. Bacteriol. 110:667. The DNA is isolated and analyzed usually by restriction enzyme analysis and/or sequencing. Sequencing may be by the well-known dideoxy method of Sanger et al. (1977) Proc. Natl. Acad. Sci. USA 74:5463) as further described by Messing et al. (1981) Nucleic Acid Res. 9:309, or by the method reported by Maxam et al. (1980) Meth. Enzymol. 65:499. Problems with band compression, which are sometimes observed in GC rich regions, are overcome by use of, for example, T-deazoguanosine or inosine, according to the method reported by Barr et al. (1986) Biotechniques 4:428.

Transfection may be by any known method for introducing polynucleotides into a host cell, including packaging the polynucleotide in a virus and transducing a host cell with the virus, by direct uptake of the polynucleotide by the host cell, and the like, which methods are known to those skilled in the art. The transfection procedures selected depend upon the host to be transfected and are determined by the rountineer.

Either a prokaryotic or a eukaryotic host cell may be used for expression of desired coding sequences when appropriate control sequences that are compatible with the designated host are used. In one embodiment the host cell is a null host cell, more preferably, when the expression of the coding sequence result in production of a P₂X purinoreceptor polypeptide, e.g., rhesus P₂X₃ of the invention, the host cell is any P₂X⁻ cell or a cell in which expression of P₂X purinoreceptor is less than can be detectably measured.

For example, among prokaryotic hosts, Escherichia coli is frequently used. Also, for example, expression control sequences for prokaryotes include but are not limited to promoters, optionally containing operator portions, and ribosome binding sites. Transfer vectors compatible with prokaryotic hosts can be derived from, for example, the plasmid pBR322 that contains operons conferring ampicillin and tetracycline resistance, and the various pUC vectors, that also contain sequences conferring antibiotic resistance markers. These markers may be used to obtain successful transformants by selection. Commonly used prokaryotic control sequences include but are not limited to the lactose operon system (Chang et al. (1977) Nature 198:1056), the tryptophan operon system (reported by Goeddel et al. (1980) Nucleic Acid Res. 8:4057) and the lambda-derived P1 promoter and N gene ribosome binding site (Shimatake et al. (1981) Nature 292:128), the hybrid Tac promoter (De Boer et al. (1983) Proc. Natl. Acad. Sci. USA 292:128) derived from sequences of the trp and lac Uv5 promoters. The foregoing systems are particularly compatible with E. coli; however, other prokaryotic hosts such as strains of Bacillus or Pseudomonas may be used if desired.

Eukaryotic hosts include yeast and mammalian cells in culture systems. Pichia pastoris, Saccharomyces cerevisiae and S. carlsbergensis are commonly used yeast hosts. Yeast-compatible vectors carry markers that permit selection of successful transformants by conferring protrophy to auxotrophic mutants or resistance to heavy metals on wild-type strains. Yeast-compatible vectors may employ the 2-.mu. origin of replication (Broach et al. (1983) Meth. Enzymol. 101:307), the combination of CEN3 and ARS1 or other means for assuring replication, such as sequences that will result in incorporation of an appropriate fragment into the host cell genome. Control sequences for yeast vectors are known in the art and include but are not limited to promoters for the synthesis of glycolytic enzymes, including the promoter for 3-phosphoglycerate kinase. See, for example, Hess et al. (1968) J. Adv. Enzyme Reg. 7:149, Holland et al. (1978) Biochemistry 17:4900 and Hitzeman (1980) J. Biol. Chem. 255:2073. For example, some useful control systems are those that comprise the glyceraldehyde-3-phosphate dehydrogenase (GAPDH) promoter or alcohol dehydrogenase (ADH) regulatable promoter, or the hybrid yeast promoter ADH2/GAPDH described in Cousens et al. Gene (1987) 61:265-275, terminators also derived from GAPDH, and, if secretion is desired, leader sequences from yeast alpha factor. In addition, the transcriptional regulatory region and the transcriptional initiation region which are operably linked may be such that they are not naturally associated in the wild-type organism.

Mammalian cell lines available as hosts for expression are known in the art and are available from depositories such as the American Type Culture Collection. These include but are not limited to HeLa cells, human embryonic kidney (HEK) cells, Chinese hamster ovary (CHO) cells, baby hamster kidney (BHK) cells, and others. Suitable promoters for mammalian cells also are known in the art and include viral promoters such as that from Simian Virus 40 (SV40), Rous sarcoma virus (RSV), adenovirus (ADV), bovine papilloma virus (BPV) and cytomegalovirus (CMV). Mammalian cells also may require terminator sequences and poly A addition sequences; enhancer sequences which increase expression also may be included, and sequences which cause amplification of the gene also may be desirable. These sequences are known in the art. Vectors suitable for replication in mammalian cells may include viral replicons, or sequences which ensure integration of the appropriate sequences encoding the P₂X receptor into the host genome. An example of such a mammalian expression system is described in Gopalakrishnan et al. (1995), Eur. J. Pharmacol.-Mol. Pharmacol. 290:237-246.

Other eukaryotic systems are also known, as are methods for introducing polynucleotides into such systems, such as amphibian cells, using standard methods such as described in Briggs et al. (1995) Neuropharmacol. 34:583-590 or Stuhmer (1992) Meth. Enzymol. 207:319-345, insect cells using methods described in Summers and Smith, Texas Agricultural Experiment Station Bulletin No. 1555 (1987), and the like.

The baculovirus expression system can be used to generate high levels of recombinant proteins in insect host cells. This system allows for high level of protein expression, while post-translationally processing the protein in a manner similar to mammalian cells. These expression systems use viral promoters that are activated following baculovirus infection to drive expression of cloned genes in the insect cells (O'Reilly et al. (1992) Baculovirus Expression Vectors: A Laboratory Manual, IRL/Oxford University Press).

Transfection may be by any known method for introducing polynucleotides into a host cell, including packaging the polynucleotide in a virus and transducing a host cell with the virus, by direct uptake of the polynucleotide by the host cell, and the like, which methods are known to those skilled in the art. The transfection procedures selected depend upon the host to be transfected and are determined by the rountineer.

The expression of the purinoreceptor, e.g., P₂X₃ may be detected by use of a radioligand selective for the receptor. For example, such ligands include ^{35S}ATP S, ^{35 S}ATP S, ^{35 S}ATP S, ^{3 H}ATP and αβmeATP (see, Michel et al. (1997) Mol. Pharmacol. 51:524-532). However, any radioligand binding technique known in the art may be used to detect the receptor (see, e.g., Winzor et al. (1995) Quantitative Characterization of Ligand Binding, Wiley-Liss, Inc., N.Y.). Alternatively, expression can be detected by utilizing antibodies or functional measurements, i.e., ATP- or UTP-stimulated cellular depolarization using methods that are well known to those skilled in the art. For example, agonist-stimulated Ca.sup.2+ influx, or inhibition by antagonists of agonist-stimulated Ca.sup.2+ influx, can be measured in mammalian cells that express endogenous and/or recombinant P2 receptor, such as HEK, CHO, COS and PC12 (rat pheochromocytoma) cells. In a particular embodiment of such methods, Ca.sup.2+ influx can be measured in cells that do not naturally express any P2 receptor (such as the 1321N1 human astrocytoma cell line) but have been prepared using recombinant technology to transiently or stably express a human P₂X₃ purinoreceptor.

In one method of expression, DNA which encodes the target P₂Xₙ purinergic receptor, e.g., P₂X₃ of SEQ ID NO.:1 or messenger RNA derived therefrom, may be introduced by direct injection into a cell such as a Xenopus laevis oocyte. Using this method, the functionality of the purinoreceptor encoded by the DNA or the mRNA can be evaluated as follows. A receptor-encoding polynucleotide is injected into an oocyte for translation into a functional receptor subunit. The function of the expressed human purinoreceptor can be assessed in the oocyte by a variety of techniques including electrophysiological techniques such as voltage-clamping (see, e.g., Briggs et al. (1995), supra) and the like.

Receptor expressed in a recombinant host cell may be used to identify compounds that modulate P₂X₃ activity. In this regard, the specificity of the binding of a compound showing affinity for the receptor is demonstrated by measuring the affinity of the compound for cells expressing the receptor or membranes from these cells. This may be done by measuring specific binding of labeled (for example, radioactive) compound to the cells, cell membranes or isolated receptor, or by measuring the ability of the compound to displace the specific binding of a standard labeled ligand. See, Michel et al., supra. Expression of variant receptor and screening for compounds that bind to, or inhibit the binding of labeled ligand to these cells or membranes, provide a method for rapid selection of compounds with high affinity for the receptor. These compounds may be agonists, antagonists or modulators of the receptor.

Expressed receptors, particularly a P₂X₃ receptor of the invention alone or in combination with P₂X₂, also may be used to screen for compounds that modulate purinoreceptor activity. One method for identifying compounds that modulate human purinoreceptor activity, comprises providing a cell that expresses a human (or, if analogous, other mammalian) purinoreceptor polypeptide, combining a test compound with the cell and measuring the effect of the test compound on the purinoreceptor activity. The cell may be a bacterial cell, a mammalian cell, a yeast cell, an amphibian cell, an insect cell or any other cell expressing the receptor. Preferably, the cell is a mammalian cell or an amphibian cell, more preferably the cell is a purinoreceptor null cell as described above. Thus, for example, a test compound is evaluated for its ability to elicit an appropriate response, e.g., the stimulation of cellular depolarization or increase in intracellular calcium levels due to calcium ion influx if a P₂X purinoreceptor is expressed in the host cell, the stimulation of an increase in intracellular calcium ion levels and/or inositolphospholipid hydrolysis and the formation of inositol phosphate if a P₂Y purinoreceptor is expressed, or for the compound's ability to modulate the response to a P₂X or P₂Y purinoreceptor agonist or antagonist.

The level of intracellular calcium may be analyzed using a calcium ion-sensitive fluorescent indicator. Cellular fluorescence may be monitored using a fluorometer. Examples of calcium ion-sensitive fluorescent dyes include, for example, quin-2 (see, e.g., Tsien et al. (1982). J. Cell. Biol. 94:325), fura-2 (see, e.g., Grynkiewicz et al. (1985) J. Biol. Chem. 260:3440), calcium green-1, indo-1 (see, e.g., Grynkiewicz et al., supra), fluo-3 (see, e.g., Kao et al. (1989) J. Biol. Chem. 264:8179) and rhod-2 (see, e.g., Tsien et al. (1987) J. Biol. Chem. abstract 89a), and the nonspecific esterase-hydrolyzable acetoxymethyl esters thereof, all of which are commercially available (Molecular Probes, Eugene, Oreg.; Sigma Chemical Co., St. Louis, Mo.).

### C. MODULATION OF P2X₃ ACTIVITY IN A CELL ACCORDING TO THE INVENTION

The discovery of ATP is a ligand of the purinoreceptor P₂, including the P₂X₃ receptor, provides methods of modulating the activity of a P₂X₃ polypeptide in a cell. P₂X₃ activity is modulated in a cell by delivering to that cell an agent that modulates the function of a P₂X₃ polypeptide. This modulation can be performed in cultured cells as part of an assay for the identification of additional modulating agents, or, for example, in an animal, including a human. Agents include ATP and its analogues as defined herein, as well as additional modulators identified using the screening methods described herein including but not limited to any of the ATP analogues presented in U.S. Pat. No. 5,700,786.

The invention thus provides for a compound that is a modulator of a receptor of the invention. As such, compounds which block or inhibit activation of P₂X₃ receptor will find use in blocking the pain stimulus. As well, antagonists to compounds which normally activate the P₂X₃ receptor and/or P₂X₂/P₂X₃ heteromeric channels, such as ATP, could successfully block the transmission of pain.

An agent can be delivered to a cell by adding it to culture medium. The amount to deliver will vary with the identity of the agent and with the purpose for which it is delivered. For example, in a culture assay to identify antagonists of P₂X₃ activity, one will preferably add an amount of ATP that half-maximally activates the receptors (e.g., approximately EC.sub.50), preferably without exceeding the dose required for receptor saturation. This dose can be determined by titrating the amount of ATP to determine the point at which further addition of ATP has no additional effect on P₂X₃ activity.

When a modulator of P₂X₃ activity is administered to an animal for the treatment of a disease or disorder, the amount administered can be adjusted by one of skill in the art on the basis of the desired outcome. Successful treatment is achieved when one or more measurable aspects of the pathology (e.g., tumor cell growth, accumulation of inflammatory cells) is changed by at least 10% relative to the value for that aspect prior to treatment.

Thus, in one embodiment of the invention, there is provided a method of screening for a candidate modulator of P₂X₃ activity using cells expressing a P₂X₃ receptor, said method compromising: a) incubating a first sample of said cells in the presence of said candidate modulator and a second sample of said cells in the absence of said candidate modulator, both said samples under conditions which permit binding of ATP to the P₂X₃ receptor; b) detecting a signaling activity of P₂X₃ receptor in said first and second samples, and c) comparing the results of said second messenger assays for said first and second samples. In general, a decrease in signaling activity would identify a potential antagonist.

Another embodiment provides a method for determining if a candidate modulator increases or decreases the activity of P₂X₃ using cells expressing a P₂X₃ receptor, said method comprising: a) incubating a first sample of said cells in the presence of said candidate modulator and a second sample of said cells in the absence of said candidate modulator, both said samples under conditions which permit binding of ATP to P₂X₃; b) detecting a signaling activity of P₂X₃ polypeptide in said first and second samples, and c) comparing the results of said second messenger assays for said first and second samples.

A method of identifying an agent that modulates the function of a P₂X₃ receptor, said method comprising: a) contacting a P₂X₃ polypeptide with ATP in the presence and absence of a candidate modulator under conditions permitting the binding of said ATP to said P₂X₃ polypeptide; and b) measuring the binding of said P₂X₃ polypeptide to said candidate modulator, relative to the binding in the absence of said candidate modulator, identifies said candidate modulator as an agent that modulates the function of P₂X₃. It is noted that other potential activators of the P₂X₃ receptor may be used in place of ATP.

In yet another embodiment, the invention provides a method of modulating a P₂X₃ receptor activity comprising the steps of: a) providing to an isolated host cell expressing a recombinant P₂X₃ receptor of sequence SEQ ID NO.:1, b) providing to said cell an amount of a P₂Xᵣ receptor-modulating molecule, e.g., ATP, sufficient either to increase or decrease a cellular response which occurs upon P₂X₃ receptor activation, wherein said molecule (1) induces one or more P₂X₃-like effects at said receptor, or (2) blocks one or more P₂X₃-like effects at said receptor. Such P₂X₃ like activities may include modulation of cystolic concentrations of cations which in the context of the present invention includes Ca²⁺, Na⁺ and K⁺. In general, according to the methods of the invention, the compound induces one or more activities selected from the group consisting of an increase in cytosolic calcium [Ca²⁺]ᵢ concentration, or an increase in cystolic Na⁺ and K⁺.

In another embodiment, the invention provides for a method of identifying a potential or candidate analgesic, the method comprising measuring the response of cells having a functional P₂X₃ receptor activity, when such cells are contacted with said compound, relative to the response of said cells to positive and/or negative control compounds.

A method for identifying a candidate analgesic comprising: contacting a cell expressing on the surface thereof a P₂X₃ receptor polypeptide, wherein the receptor is associated with a second component capable of providing a detectable signal in response to the binding of a compound to the receptor, with a compound to be screened under conditions favoring binding of the compound to the polypeptide; and determining whether the compound binds to and activates or inhibits the receptor polypeptide by measuring the level of a signal generated from the interaction of the compound with the receptor polypeptide.

In yet another embodiment, the invention provides a method for determining whether a compound has agonist or antagonist activity relative to a P₂X₃ receptor polypeptide, the method comprising determining the effect of a candidate compound on the influx of calcium ions into cells known to express the P₂X₃ receptor polypeptide, relative to the rate of influx of calcium ions into such cells contacted with positive and negative control compounds. In one example, the control population of cells express either a null cell, a cell that does not express the P₂X₃ receptor or one the expresses a dysfunctional P₂Xₙ receptor or one where cells expressing a functional P₂Xᵣ receptor are not contacted with said candidate compound. Thus, in one example, the decrease in calcium influx is evaluated by determining such influx in a test population, e.g., P₂X₃ expressing cells contacted with the test compound compared to a control population wherein identical cells are not contacted with the rest compound. In circumstances, wherein a decrease in calcium ion influx in the test population of cells, e.g., cells incubated in the presence of said test compound relative to calcium influx in a control sample of cells, e.g., those that are incubated in the absence of the test compound, is an indicator of P₂X₃ antagonist activity in the test compound.

Consequently, a general method for identifying a potential analgesic or antagonist compound, e.g., one that is an antagonist of the P₂Xᵣ receptor polypeptide, comprise: a) contacting recombinant cells expressing a functional P₂X₃ receptor with a test compound, and b) measuring ion flux, an electrophysiological response of the cells, or binding of the test compound to the P₂Xᵣ receptor polypeptide, whereby agonist or antagonists of the P₂X₃ receptor are identified, by measuring any one of more of the aforementioned parameters relative to control, such that a decrease relative to normal, will, in general identify a potential antagonist, while an increase in said parameter will likely identify a potential agonist.

Membrane depolarization of cells genetically engineered to express a P₂Xₙ purinoreceptor, e.g., P₂X₃ may be monitored using a fluorescent dye that is sensitive to changes in membrane potential. For example, the potential-sensitive fluorescent dye partitions into a membrane upon depolarization and results in a detectable increase in cellular fluorescence. Examples of such membrane potential-sensitive fluorescent dyes include carbocyanines, such as 3,3′-dipentyloxacarbocyanine iodide (DiOC.sub.5) and 3,3′-dipropylthiadicarbocyanine iodide (DiSC.sub.3), oxonols, such as bis-(1,3-dibutylbarbituric acid)pentamethine oxonol (DiBAC.sub.4 (5)) or bis-(1,3-dibutylbarbituric acid)pentamethine oxonol (DiBAC.sub.4 (5)), or the like.

In order to calibrate the fluorescence emission of these dyes in situ, an agent that quenches the fluorescence emission may, be used. Thus, for example, anti-fluorescein (Molecular Probes) quenches approximately 87% of the fluorescence of a 5 nM solution of fluo-3 at pH 7.0, and may used to calibrate the fluorescence emission of this dye. When acetoxymethyl ester dye derivatives are use, incomplete hydrolysis of the ester may result in a fluorescent indicator that is flourescent but insensitive to calcium ions. Controls for such a situation include transporting saturating amounts of calcium ions into the cell by an ionophore to achieve the maximum fluorescence response and transport of manganese ions into the cell to quench the fluorescence of the indicator if all acetoxymethyl esters have been hydrolyzed. One means by which such ions can be transported into cells is with the use of an ionophore, such as A23187 (see, e.g., Pressman et al. (1976) Ann. Rev. Biochem. 45:501) (Sigma Chemical Co.), the brominated derivative thereof (see, e.g., Deber et al. (1985) Anal. Biochem. 146:349) (Molecular Probes), or other ionophores well known in the art.

In addition, it may be desirable to quantify the amount of intracellular calcium ion from the fluorescence emission of a cell by comparing the fluorescence data obtained from the test compounds to a calibration curve that was generating by a series of calibrators each having a known calcium ion concentration. Thus, calcium ion standards are made having a range of concentrations by preparing a stock solution of, e.g., CaCl.sub.2, from which dilutions may be made to attain the desired standard concentration(s). The fluorescence emission of the standards in the presence of the calcium ion-sensitive fluorescent indicator dye is used to construct a standard curve and the intracellular calcium ion concentration of the genetically engineered cell in the assay is determined from the standard curve. Alternatively, cells previously treated with a calcium ionophore may be incubated with the indicator dye and the calcium ion standards used to generate the standard curve.

The assay may be conducted manually or using an automated system. For a high capacity functional screening assay identifying human purinoreceptor ligands, an automated system is preferred. An example of such an automated system comprises providing a 96, 384, or 1536-well culture plate in each well of which is cultured a cell genetically engineered to encode and express a human purinoreceptor polypeptide. The plate is loaded into a fluorescence imaging plate reader (“FLIPR”), which simultaneously measures the kinetics of intracellular calcium flux in each of the wells. Such an FLIPR is commercially available from Molecular Devices Corp. (Sunnyvale, Calif.). The FLIPR is capable of quantitatively transferring fluids into and from each well of the plate and thus can be used to add the calcium-ion sensitive fluorescent indicator dye, a candidate compound, a purinoreceptor agonist, e.g., ATP, UTP, 2-methylthioATP, or the like, and/or a purinoreceptor antagonist, e.g., suramin, cibacron blue, PPADS, or the like. The FLIPR collects fluorescence data throughout the course of the assay.

In a similar manner, the presence of a purinoreceptor agonist or antagonist in a test sample may be determined using a manual or an automated system. An automated system for practicing the method comprises providing a 96, 384, or 1536-well culture plate in each well of which a genetically engineered cell that expresses a purinoreceptor is cultured. The fluorescent indicator dye, test sample, and/or purinoreceptor agonist are added to each well and the fluorescence emission from each well is simultaneously monitored by an FLIPR.

Transcriptional Reporters for Downstream Pathway Activation—The intracellular signal initiated by binding of an agonist to a receptor, e.g., P₂X₃, sets in motion a cascade of intracellular events, the ultimate consequence of which is a rapid and detectable change in the transcription or translation of one or more genes. The activity of the receptor can therefore be monitored by detecting the expression of a reporter gene driven by control sequences responsive to P₂X₃ activation. As such, conspicuously comprehended herein are reporter gene assays.

Reporter genes such as luciferase, CAT, GFP, .beta.-lactamase or .beta.-galactosidase are well known in the art, as are assays for the detection of their products.

Genes particularly well suited for monitoring receptor activity are the “immediate early” genes, which are rapidly induced, generally within minutes of contact between the receptor and the effector protein or ligand. The induction of immediate early gene transcription does not require the synthesis of new regulatory proteins. In addition to rapid responsiveness to ligand binding, characteristics of preferred genes useful for making reporter constructs include: low or undetectable expression in quiescent cells; induction that is transient and independent of new protein synthesis; subsequent shut-off of transcription requires new protein synthesis; and mRNAs transcribed from these genes have a short half-life. It is preferred, but not necessary that a transcriptional control element have all of these properties for it to be useful.

An example of a gene that is responsive to a number of different stimuli is the c-fos proto-oncogene. The c-fos gene is activated in a protein-synthesis-independent manner by growth factors, hormones, differentiation-specific agents, stress, and other known inducers of cell surface proteins. The induction of c-fos expression is extremely rapid, often occurring within minutes of receptor stimulation.

This characteristic makes the c-fos regulatory regions particularly attractive for use as a reporter of receptor activation.

The c-fos regulatory elements include a TATA box that is required for transcription initiation; two upstream elements for basal transcription, and an enhancer, which includes an element with dyad symmetry and which is required for induction by TPA, serum, EGF, and PMA. See, Verma et al., 1987, Cell 51: 513-514.

The 20 by c-fos transcriptional enhancer element located between −317 and −298 by upstream from the c-fos mRNA cap site, is essential for serum induction in serum starved NIH 3T3 cells. One of the two upstream elements is located at −63 to −57 and it resembles the consensus sequence for cAMP regulation.

The transcription factor CREB (cyclic AMP responsive element binding protein) is, as the name implies, responsive to levels of intracellular cAMP. Therefore, the activation of a receptor that signals via modulation of cAMP levels can be monitored by detecting either the binding of the transcription factor, or the expression of a reporter gene linked to a CREB-binding element (termed the CRE, or cAMP response element). The DNA sequence of the CRE is TGACGTCA. Reporter constructs responsive to CREB binding activity are described in U.S. Pat. No. 5,919,649.

Other promoters and transcriptional control elements, in addition to the c-fos elements and CREB-responsive constructs, include the vasoactive intestinal peptide (VIP) gene promoter (cAMP responsive; Fink et al., 1988, Proc. Natl. Acad. Sci. 85:6662-6666); the somatostatin gene promoter (cAMP responsive; Montminy et al., 1986, Proc. Natl. Acad. Sci. 8.3:6682-6686); the proenkephalin promoter (responsive to cAMP, nicotinic agonists, and phorbol esters; Comb et al., 1986, Nature 323:353-356); the phosphoenolpyruvate carboxy-kinase (PEPCK) gene promoter (cAMP responsive; Short et al., 1986, J. Biol. Chem. 261:9721-9726).

Additional examples of transcriptional control elements that are responsive to changes in GPCR activity include, but are not limited to those responsive to the AP-1 transcription factor and those responsive to NF-KB activity. The consensus AP-1 binding site is the palindrome TGA(C/G)TCA (SEQ ID NO.: 5) (Lee et al., 1987, Nature 325: 368-372; Lee et al., 1987, Cell 49: 741-752). The AP-1 site is also responsible for mediating induction by tumor promoters such as the phorbol ester 12-O-tetradecanoylphorbol-.beta.-acetate (TPA), and are therefore sometimes also referred to as a TRE, for TPA-response element. AP-1 activates numerous genes that are involved in the early response of cells to growth stimuli. Examples of AP-1-responsive genes include, but are not limited to the genes for Fos and Jun (which proteins themselves make up AP-1 activity), Fos-related antigens (Fra) 1 and 2, I.kappa.B.alpha., ornithine decarboxylase, and annexins I and II.

The NF-.kappa.B binding element has the consensus sequence GGGGACTTTCC (SEQ ID NO.:6). A large number of genes have been identified as NF-.kappa.B responsive, and their control elements can be linked to a reporter gene to monitor GPCR activity. A small sample of the genes responsive to NF-.kappa.B includes those encoding IL-1.beta. (Hiscott et al., 1993, Mol. Cell. Biol. 13: 6231-6240), TNF-.alpha. (Shakhov et al., 1990, J. Exp. Med. 171: 35-47), CCR5 (Liu et al., 1998, AIDS Res. Hum. Retroviruses 14: 1509-1519), P-selection (Pan & McEver, 1995, J. Biol. Chem. 270: 23077-23083), Fas ligand (Matsui et al., 1998, J. Immunol. 161: 3469-3473), GM-CSF (Schreck & Baeuerle, 1990, Mol. Cell. Biol. 10: 1281-1286) and I.kappa.B.alpha. (Haskill et al., 1991, Cell 65: 1281-1289).

Each of these references is incorporated herein by reference. Vectors encoding NF-.kappa.B-responsive reporters are also known in the art or can be readily made by one of skill in the art using, for example, synthetic NF-.kappa.B elements and a minimal promoter, or using the NF-.kappa.B-responsive sequences of a gene known to be subject to NF-.kappa.B regulation. Further, NF-.kappa.B responsive reporter constructs are commercially available from, for example, CLONTECH.

A given promoter construct should be tested by exposing P₂X₃-expressing cells, transfected with the construct, to ATP. An increase of at least two-fold in the expression of reporter in response to ATP indicates that the reporter is an indicator of P₂X₃ activity.

In order to assay P₂X₃ activity with an ATP responsive transcriptional reporter construct, cells that stably express a P₂X₃ polypeptide are stably transfected with the reporter construct. To screen for agonists, the cells are left untreated, exposed to candidate modulators, or exposed to ATP, and expression of the reporter is measured. The ATP-treated cultures serve as a standard for the level of transcription induced by a known agonist. An increase of at least 50% in reporter expression in the presence of a candidate modulator indicates that the candidate is a modulator of P₂X₃ activity. An agonist will induce at least as much, and preferably the same amount or more, reporter expression than ATP alone. This approach can also be used to screen for inverse agonists where cells express a P₂X₃ polypeptide at levels such that there is an elevated basal activity of the reporter in the absence of ATP or another agonist. A decrease in reporter activity of 10% or more in the presence of a candidate modulator, relative to its absence, indicates that the compound is an inverse agonist.

To screen for antagonists, the cells expressing P₂X₃ and carrying the reporter construct are exposed to ATP (or another agonist) in the presence and absence of candidate modulator. A decrease of 10% or more in reporter expression in the presence of candidate modulator, relative to the absence of the candidate modulator, indicates that the candidate is a modulator of P₂X₃ activity.

Controls for transcription assays include cells not expressing P₂X₃ but carrying the reporter construct, as well as cells with a promoterless reporter construct. Compounds that are identified as modulators of P₂X₃-regulated transcription should also be analyzed to determine whether they affect transcription driven by other regulatory sequences and by other receptors, in order to determine the specificity and spectrum of their activity.

The transcriptional reporter assay, and most cell-based assays, are well suited for screening expression libraries for proteins for those that modulate P₂X₃ activity. The libraries can be, for example, cDNA libraries from natural sources, or they can be libraries expressing randomly or systematically mutated variants of one or more polypeptides. Genomic libraries in viral vectors can also be used to express the mRNA content of one cell or tissue, in the different libraries used for screening of P₂X₃.

Compounds capable of modulating P₂X₃ receptor are considered potential therapeutic agents in several disorders including, without limitation, central nervous system or peripheral nervous system conditions, for example, epilepsy, pain, depression, neurodegenerative diseases, and the like, and in disorders of skeletal muscle such as neuromuscular diseases.

In addition, the DNA, or RNA derived therefrom, can be used to design oligonucleotide probes for DNAs that express specific P₂X receptor. As used herein, the term “probe” refers to a structure comprised of a polynucleotide, as defined above, which contains a nucleic acid sequence complementary to a nucleic acid sequence present in a target polynucleotide. The polynucleotide regions of probes may be composed of DNA, and/or RNA, and/or synthetic nucleotide analogs. Such probes could be useful in in vitro hybridization assays to distinguish P₂X₃ variants from wild-type message, with the proviso that it may be difficult to design a method capable of making such a distinction given the small differences that may exist between sequences coding the wild-type and a variant P₂X receptor. Alternatively, a PCR-based assay could be used to amplify the sample RNA or DNA for sequence analysis.

Furthermore, each specific P₂X polypeptide or fragment(s) thereof can be used to prepare monoclonal antibodies using techniques that are well known in the art. The specific P₂X receptor or relevant fragments can be obtained using the recombinant technology outlined below, i.e., a recombinant cell that expresses the receptor or fragments can be cultured to produce quantities of the receptor or fragment that can be recovered and isolated. Alternatively, the specific P₂X polypeptide or fragment(s) thereof can be synthesized using conventional polypeptide synthetic techniques as known in the art. Monoclonal antibodies that display specificity and selectivity for a particular P₂X polypeptide can be labeled with a measurable and detectable moiety, for example, a fluorescent moiety, radiolabels, enzymes, chemiluminescent labels and the like, and used in in-vitro assays. It is theorized that such antibodies could be used to identify wild-type or variant P₂X receptor polypeptides for immuno-diagnostic purposes. For example, antibodies have been generated to detect amyloid b1-40 v. 1-42 in brain tissue (Wisniewski et al. (1996) Biochem. J. 313:575-580; also see, Suzuki et al. (1994) Science 264:1336-1340; Gravina et al. (1995) J. Biol. Chem. 270:7013-7016; and Turret et al. (1996) J. Biol. Chem. 27′1:8966-8970).

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### Example 1

Rhesus P₂X₃ cDNA Cloning—A full-length rhesus P₂X₃ receptor cDNA was cloned from rhesus dorsal root ganglion (DRG) cDNA using the polymerase chain reaction (PCR). The PCR primers were based upon the human P₂X₃ receptor (5′-GAAGCTTACCATGAACTGCATATCC-3′ (SEQ ID NO.:3) and 5′-GCTCGAGCTAGTGGCCTATGGAGAAG-3′ (SEQ ID NO.:4)) and contained 5′HindIII and 3′XhoI restriction sites to facilitate expression vector construction. Amplification reactions consisted of 35 cycles of 30 sec at 94° C., 30 sec at 57° C., and 2 min at 70° C. and were carried out according to the manufacturer's recommended protocol for Platinum PCR SuperMix High Fidelity (Invitrogen). Multiple sublcones were sequenced to rule out potential PCR errors.

Generation of a Rhesus P₂X₃ Stable Cell Line—Rhesus P₂X₃ receptor cDNA was subcloned as a 5′HindIII and 3′XhoI fragment into the expression vector pcDNA5/FRT/TO (Invitrogen). Five micrograms of the rhesus P₂X₃ expression construct was transfected using Lipofectamine 2000 (Invitrogen) into Flp-in T-Rex—293 cells (Invitrogen) according to the manufacturer's directions. Cells positive for flp-mediated recombination of rhesus P₂X₃ were selected using 300 μg/ml hygromycin. The stable rhesus P₂X₃ cell line was propagated in DMEM, 10% FBS, 150 μg/ml hygromycin, 15 μg/ml blasticidin, and 100 units/ml penicillin and 100 μg/ml streptomycin, and maintained at 37° and 95% humidity. Cells were subcultured by treatment with 0.25% trypsin with 0.1% EDTA in HBSS.

Intracellular Calcium Measurement to Assess Antagonist Affinity—A fluorescent imaging plate reader (FLIPR; Molecular Devices) was used to monitor intracellular calcium levels using the calcium-chelating dye Fluo-3 (Molecular Probes). Cells expressing the rhesus P₂X₃ receptor were plated in the presence of 0.5 to 1 μg/ml doxycycline at a density of 30,000 cells/well in 384-well black-walled plates approximately 20 hours before beginning the assay. Cells were washed with HBSS and dye-loaded in HBSS, 2 mM CaCl₂, 0.1% BSA, 0.25 mM probenecid, and 4 μM Fluo-3 for 60 min in the dark at room temperature. Prior to compound additions cells were washed with HBSS. Antagonist affinity was determined by the ability of these compounds to inhibit increases of intracellular calcium elicited by the P₂X₃ agonist αβ-meATP. Ten minutes prior to adding agonist, the antagonist was added and allowed to incubate at room temperature. Following agonist addition fluorescence data was collected at 5 sec intervals and analyzed based on the increase in peak relative fluorescence units (RFU) compared to the basal fluorescence. Antagonist dose-response data expressed as a response ratio (max RFU/min RFU) were analyzed using GraphPad Prism.

In vitro Electrophysiological Assay—Cells expressing rhesus P₂X₃ receptor were grown to a confluence of 65-85% and induced with 1 μg/ml doxycycline 20 to 32 hours prior to assay. After induction the cells were dissociated with trypsin, centrifuged, and resuspended in bath solution at a cell density of 6×10⁶ cells/ml and loaded onto PatchXpress. The bath solution contained 150 mM NaCl, 4 mM KCl, 2 mM CaCl₂, 1.2 mM MgCl₂, 10 mM HEPES, and 11.1 mM glucose, at pH 7.2. The intracellular solution contained 140 mM K-aspartate, 20 mM NaCl, 5 mM HEPES, 10 mM EGTA, at pH 7.2. Agonist stock solutions were prepared in H₂O and diluted in bath solution prior to use. All antagonists were prepared as 10 mM stock solutions in DMSO and diluted in bath solution prior to use. All experiments were performed on cells under the whole-cell patch clamp configuration at room temperature. Up to 16 individual cells could be patch clamped simultaneously on the PatchXpress instrument. A baseline response was established by repeated CTP (100 μM; for 2 sec.) followed by antagonist incubation for 2 min. in the absence of CTP. After antagonist preincubation 100 μM CTP and antagonist were co-administered to determine the inhibitory effect of the antagonist. These steps were then repeated on the same cell with a range of concentrations of the antagonist. A maximum of five concentrations of antagonist were tested on any individual cell. The control P₂X₃ current amplitude (I_{P2X3-(control)}) was taken as an average of the peak current amplitude from the last two agonist additions prior to incubation with an antagonist. The peak P₂X₃ current amplitude in the presence of an antagonist (I_{P2X3-(drug)}) was used to calculate the inhibitory effect at each concentration of the antagonist according to the following equation:

% inhibition of P₂X₃=100*(I_{P2X3-(control)}−I_{P2X3-(drug)})/I_{P2X3-(control)}

Each concentration of an antagonist was tested on at least three independent cells. The concentration of drug required to inhibit P₂X₃ current by 50% (IC₅₀) was determined by fitting of the Hill equation to the averaged % inhibition data at each concentration:

% of Control=100·(1+([Drug]/IC₅₀)^{p})⁻¹

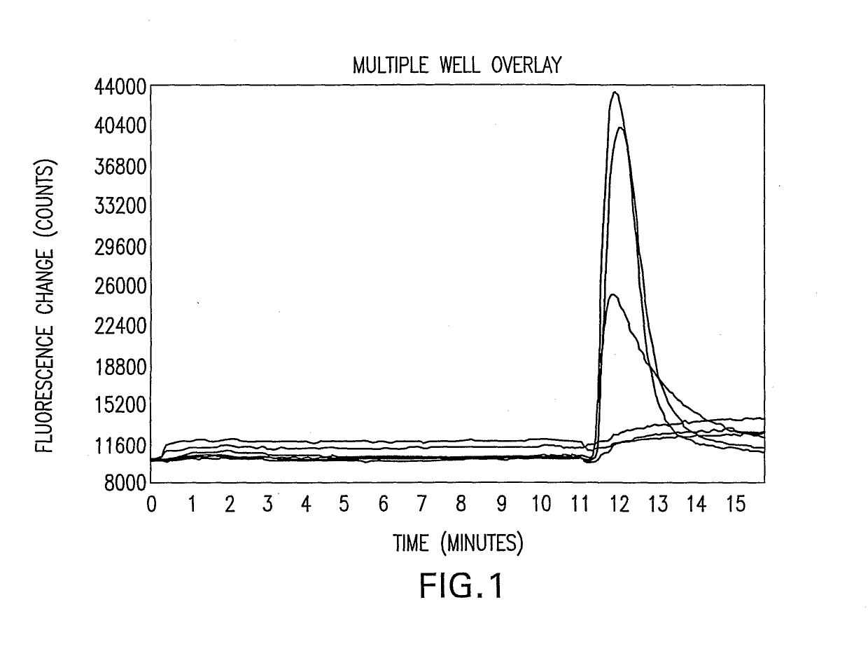

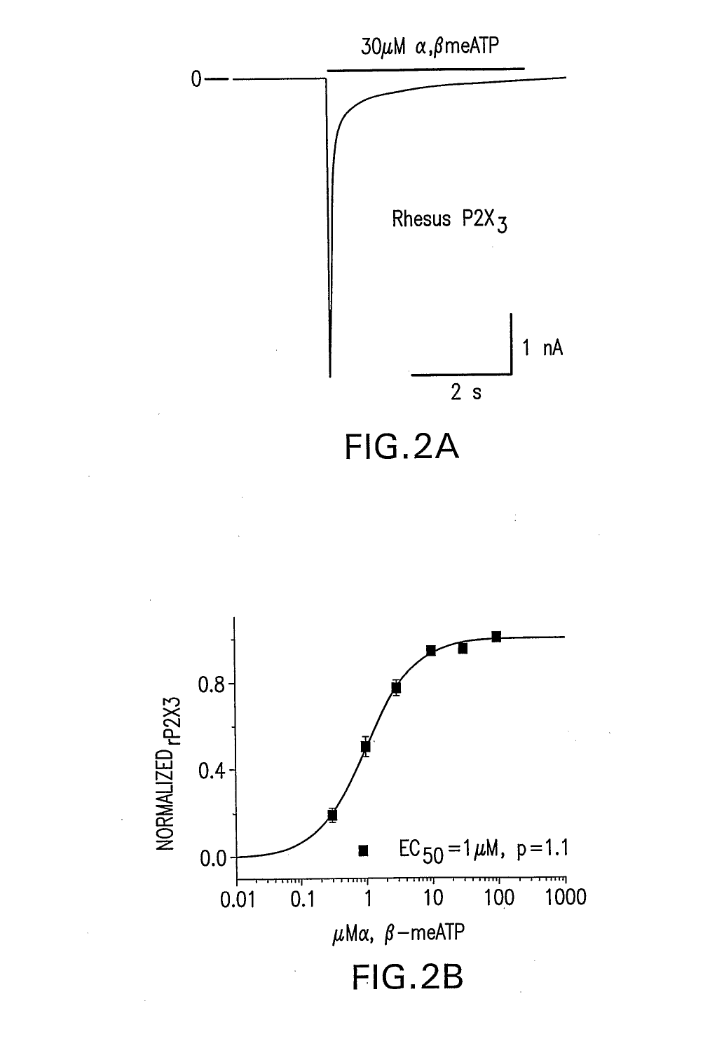

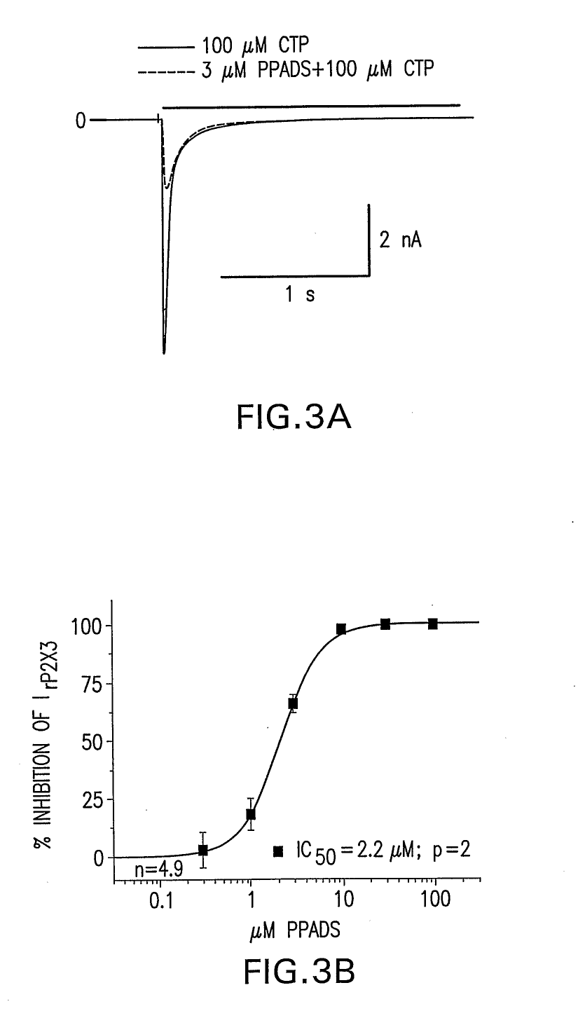

Results—The rhesus ortholog of the human P₂X₃ receptor was cloned and found to exhibit 97.4% and 98.99% identity to the human receptor on the nucleotide and peptide level, respectively. A rhesus P₂X₃ stable cell line was generated and profiled in two in vitro functional assays. Using a fluorescent imaging plate reader the potency of two known P2X antagonists, suramin and pyridoxal-phoshpate-6-azophenyl-2′,4′-disulfonic acid (PPADS), were evaluated. Both suramin and PPADS (FIG. 1) dose-dependently blocked the a0-meATP mediated increase in intracellular calcium with an IC₅₀ of 1.5 μM and 2.9 μM for suramin and PPADS, respectively. An inward current was evoked by the application of the P₂X agonist αβ-meATP with an EC₅₀ of 1 μM measured via patch-clamp techniques (FIG. 2). Additionally, using an in vitro electrophysiological assay suramin and PPADS (FIG. 3) were shown to dose-dependently inhibit CTP-induced currents with an 10₅₀ of 382 nM and 2.2 μM for suramin and PPADS, respectively. In conclusion, the rhesus P₂X₃ receptor represents a valid alternative to the human receptor for the evaluation of P₂X₃ receptor antagonists.

1.-15. (canceled)

16. An isolated polynucleotide encoding a rhesus P₂X₃ receptor protein comprising the amino acid sequence of SEQ ID NO.:2.

17. An isolated polynucleotide encoding a receptor protein of claim 16, or a functionally active fragment thereof, wherein said polynucleotide comprises a sequence selected from the group consisting of:
(a) a sequence of nucleotides that encodes the amino acid sequence set forth in SEQ ID NO.:2, or a functionally active fragment thereof;
(b) a sequence of nucleotides as set forth in SEQ ID NO.:1; and
(c) a sequence of nucleotides that encode a mammalian purinergic receptor subtype and that hybridizes to the sequence of nucleotides of (a) or (b) under moderately stringent conditions, wherein said sequence of nucleotides encodes a biologically active mammalian purinergic receptor subtype designated as P₂X₃ herein.

18. An expression vector comprising a polynucleotide of claim 17.

19. A host cell transformed or transfected with the expression vector of claim 18.

20. A host cell of claim 19, wherein said cell is selected from the group consisting of a bacterial cell, a mammalian cell, a yeast cell and an amphibian cell.

21. A host cell according to claim 20, wherein the cell is an amphibian cell.

22. A host cell according to claim 20, wherein the cell is a mammalian cell.

23. A method for producing a rhesus P₂X₃ receptor polypeptide, the method comprising the steps of:
(a) transfecting or transforming a suitable host cell with the expression vector of claim 18;
(b) culturing the host cells of step (a) under conditions which allow expression of the recombinant receptor polypeptide from the expression vector; and
(c) harvesting the recombinant receptor polypeptide from the culture, culturing a host.
